(19) United States
(12) Patent Application Publication
(10) Pub. No.: US 2007/0031875 A1
(43) Pub. Date: Feb. 8, 2007

(54) Signal pattern compositions and methods

(75) Inventor: Philip Richard Buzby, Brockton, MA (US)

Correspondence Address: SULLIVAN & WORCESTER LLP, ONE POST OFFICE SQUARE, BOSTON, MA, 02109, US

(73) Assignee: Helicos BioSciences Corporation, Cambridge, MA (US)

(21) Appl. No.: 11/500,165

(22) Filed: Aug. 7, 2006

Related U.S. Application Data
(60) Provisional application No. 60/705,941, filed on Aug. 5, 2005.

(51) Int. Cl. C12Q 1/68; C07H 21/02

(52) U.S. Cl. 435/6; 536/25.32

(57) ABSTRACT

The invention relates to compositions and methods for a temporal signal pattern or signature representative of a characteristic of a nucleic acid template. The signature is created by monitoring the real time sequential pattern of signal emissions generated during nucleic acid template-dependent polymerization reactions.

### CROSS-REFERENCE TO RELATED APPLICATION

This application claims priority to U.S. Provisional Patent Application No. 60/705,941, filed on Aug. 5, 2005.

### TECHNICAL FIELD OF THE INVENTION

The invention relates to compositions and methods for a signal pattern or signature representative of a characteristic of a nucleic acid template.

### BACKGROUND OF THE INVENTION

The completion of a consensus human genome sequence has prompted inquiry into genetic differences within and between individuals as the basis for differences in biological function and dysfunction. For example, single nucleotide differences between individuals that give rise to single nucleotide polymorphisms (SNPs) may result in dramatic phenotypic differences. Those differences may be manifested in outward expressions of altered phenotype, may determine the likelihood that an individual will get a certain disease, or may determine how an individual will respond to a particular treatment. For example, most cancers develop from a series of genomic changes, some subtle and some major, that occur in a small subpopulation of cells. Knowledge of the sequence variations that lead to cancer will foster an understanding of the etiology of the disease, as well as ways to treat and prevent it. Furthermore, detection and identification of SNPs and other genomic variations is useful in the fields of epidemiology, infectious disease, and forensics, among others.

An essential step in understanding genomic complexities is the ability to perform rapid, high-resolution nucleic acid characterization without undertaking whole- or partial-genome nucleic acid sequencing. For example, certain research and diagnostic methods identify variant nucleic acid sequences, such as mutations or polymorphisms, without the need for direct sequencing. These methods include one base scanning (OBS); base excision signal sequencing (BESS; Epicentre, Madison, Wis.); restriction fragment length polymorphism (RFLP) analysis; amplification fragment length polymorphism (AFLP) analysis; variable number tandem repeat (VNTR) analysis; short tandem repeat (STR) analysis; single-stranded conformational polymorphism (SSCP) analysis; heteroduplex analysis; and random amplification of polymorphic DNA (RAPD). Such methods are useful in a variety of applications, including mutation or polymorphism detection for disease diagnosis; forensics; paternity or maternity testing; population genetics; genetic linkage analysis; taxonomy; transcript identification; and pharmacogenomics; and microorganism detection, identification, diagnosis, and tracking. For example, RFLP analysis (also referred to as DNA-fingerprinting) is used in forensics to compare nucleic acid samples of unknown sequence or identity to a known nucleic acid sample. SSCP analysis is used to identify changes in electrophoretic migration patterns of small nucleic acid fragments that are indicative of sequence variations. VNTR and STR analyses are used to identify individual differences in the number of nucleotide repeats present at a certain locus or at a plurality of loci and are used in linkage studies, paternity testing, and forensics.

The above exemplary electrophoresis-based methods and related techniques generate variably spaced band patterns that resemble bar codes, without providing complete sequence information. These methods have limited use and reliability, however, since they generally also require the use of sequencing gels and apparatus, automated sequencers, and/or bulk nucleic acid preparation (e.g., amplification), which can introduce error, and are time consuming and costly, making data assessment inaccurate, inefficient, and/or impractical.

A need therefore exists for diagnostic and/or analytical compositions and methods that provide a signature signal pattern representative of a nucleic acid template that do not require bulk nucleic acid preparation and/or gel electrophoresis.

### SUMMARY OF THE INVENTION

The invention provides compositions and methods that provide a signal pattern or signature generated by the sequence of template-dependent base additions to a nucleic acid template/primer duplex. Compositions and methods of the invention utilize template-dependent polymerization to generate information about a nucleic acid by monitoring nucleotide incorporation over multiple incorporation cycles. Although the invention may be practiced using bulk nucleic acid sequencing, the invention is especially useful at the single molecule level because it can provide information on single nucleic acid molecules, thereby eliminating the need for amplification or other bulk nucleic acid preparation. According to the invention, each of the four common nucleotide triphosphates (or analogs) is exposed to a template/primer duplex in the presence of polymerase under conditions in which template-dependent nucleotide incorporation into the primer will occur. One, two, or three of the four nucleotide triphosphates is detectably labeled. Template-dependent nucleotide addition to the primer is allowed to occur. Upon each cycle of incorporation (the addition of a nucleotide to the 3′ end of the primer), the presence or absence of signal is noted. The presence of a signal in an incorporation cycle indicates that a labeled nucleotide has been incorporated. The absence of signal in any cycle means that a non-labeled nucleotide has been incorporated (because all four nucleotides are present). The pattern of signal detected over multiple incorporation cycles represents a unique signature of the template nucleic acid being interrogated. According to methods of the invention, it is not necessary to identify the species of nucleotide being incorporated, as the “on-off” pattern of signal detection provides a unique signature.

In one aspect, the invention provides methods that comprise generating a signal pattern representative of nucleotide incorporations into nucleic acid template/primer duplex. A nucleic acid may be identified, distinguished, or compared to another nucleic acid according to the unique signal pattern generated during the template-dependent synthesis reaction.

Methods of the invention comprise exposing a target nucleic acid to a primer, a polymerase, and at least one but fewer than four labeled nucleotides in the presence of one or more unlabeled nucleotides, whereby the polymerase incorporates a certain labeled or unlabeled nucleotide into the primer strand if a complementary nucleotide is present in the target nucleic acid. In an embodiment, signals associated with incorporated nucleotides are detected in real time, wherein incorporation of labeled and unlabeled nucleotides occurs according to the nucleotide sequence of the nucleic acid template. For example, the presence or absence of a fluorescent signal associated with successive template-dependent incorporations is compiled, producing a signal pattern that is characteristic of the specific nucleic acid template. Thus, if optically-detectable labels are used, a characteristic pattern comprising light and dark, and/or color, is generated. This characteristic pattern may be compared to a pattern generated by other nucleic acid templates or may be associated with a particular phenotype, such as a disease state, or may be used to identify or verify a drug target. Other uses of the technology described herein are possible, such as comparative genomic analysis, and others known in the art.

In practice of the invention, nucleotides may be added to the polymerase reaction sequentially or they may be added all at once to the reaction mixture. For a sequential addition, in an embodiment, the incorporation may be monitored in real time to provide a temporal signal pattern of light and dark signals. In one embodiment, the labels attached to the nucleotides to be added are of different colors and the temporal pattern comprises variation in color. In another embodiment, the temporal pattern comprises a combination of light, dark, and or color signals. Each nucleotide addition of the temporal signal pattern may be separately detected or an overall temporal pattern of periods of light or dark or color may be detected. Alternatively, non-optical labels are used and the pattern generated is based upon the detection of the non-optical labels in sequence on the primer.

In one embodiment, the invention comprises conducting template-dependent nucleic acid polymerization reactions on a nucleic acid that is attached to a solid support. In an embodiment, a primer/template duplex is attached to a surface such that the duplex is individually optically resolvable. A primer may be hybridized to a nucleic acid template to form a primer/template duplex of the invention before or after attachment to a surface. In one embodiment, a nucleic acid is exposed to a primer in solution for hybridization and the resulting primer/template duplex is deposited on a solid support. In another embodiment, a nucleic acid is exposed to the surface of a solid support and then “captured” by a complementary primer. A primer of the invention may comprise an oligonucleotide having a particular sequence, or the primer may comprise a mixture of oligonucleotides having different or random sequences.

Compositions of the invention comprise a “bar code” or signal pattern that is useful as a signature for a specific nucleic acid template. In an embodiment, the compositions of the invention comprise a series of light and dark signals or variably-colored signals, or a combination thereof, that correspond to a feature of a nucleic acid template. In one embodiment, the feature is a mutation or a polymorphism. In another embodiment, the bar code is used to distinguish between two or more nucleic acids.

Compositions of the invention may include more than one feature of a nucleic acid. For example, a signal pattern of the invention may simultaneously contain information about both polymorphisms and mutations. In some embodiments, elucidation of a collection of characteristics in a signal pattern provides a distinct feature of a nucleic acid. In one embodiment, compositions of the invention are used to distinguish between two nucleic acids. In another embodiment, compositions of the invention are used to discover new genetic events in a nucleic acid by comparing two nucleic acids. In other embodiments, comparison of two signal patterns confirms that two nucleic acid templates comprise identical sequences.

Compositions and methods of the invention contemplate generating signal patterns for any type of nucleic acid template, for example, genomic DNA, cDNA, or RNA. The nucleic acids may be isolated from an animal tissue, an animal fluid, a plant tissue, a plant fluid, an insect or insect fluid, or a microorganism or other infectious agent, for example. In one embodiment, a nucleic acid is isolated from a human tissue or fluid, such as blood. In an embodiment, the signal pattern for a test sample is compared to a signal pattern generated from a control or reference sample, such as a normal or non-disease-related nucleic acid and the presence of genetic differences between the two nucleic acids is determined. In another embodiment, the temporal signal pattern is compared to a signal pattern generated from an abnormal or diseased-related nucleic acid.

Nucleotide species useful in the practice of the invention include dATP, dCTP, dTTP, dGTP, or dUTP, natural or non-natural nucleotides, nucleotide analogs, di-deoxy nucleotides, or other nucleotide or functional equivalent that can be incorporated into a primer strand during template-dependent nucleic acid polymerization.

Nucleotides for use in the invention may be labeled with any detectable label. In a preferred embodiment, labeled nucleotides of the invention comprise an opticallydetectable label. In an embodiment, the label is a fluorescent label, such as, for example, fluorescein, rhodamine, phosphor, polymethadine dye, fluorescent phosphoramidite, texas red, green fluorescent protein, acridine, cyanine, cyanine-5 dye, cyanine-3 dye, 5-(2′-aminoethyl)-aminonaphthalene-1-sulfonic acid (EDANS), BODIPY, ALEXA, or a derivative or modification of any of the foregoing. Preferably, labels are attached via linkers, preferably cleavable linkers, such as disulfide linkers.

In a preferred embodiment of the invention, a signal pattern is generated by monitoring incorporation of one or more optically-labeled nucleotides in real time. In certain embodiments, the optically-detectable label is present on one, two, or three, but not four of the species of nucleotides. The relative number of labeled and unlabeled nucleotides depends upon the number of different nucleotides in the template. For example, if the template only comprises two species of nucleotide (e.g., only adenine and thymine) then only one unlabeled nucleotide is used. If all four nucleotides are present in the nucleic acid template (e.g., A, G, C, and T) then all four nucleotides should be present, at least one of which should be unlabeled, so that processivity is not interrupted and a pattern of “light” and “dark” will result. In a certain embodiment, one species of nucleotide is labeled and is added to the polymerization reaction in the presence of the other three unlabeled species of nucleotides. In another embodiment, two species of nucleotides are labeled and are added to the polymerization reaction in the presence of two other unlabeled species of nucleotides. In another embodiment, three species of nucleotides are labeled and are added to the polymerization reaction in the presence of the other unlabeled species of nucleotide. In another embodiment, the nucleotides are labeled with different labels and the differences between the labels are detected in addition to or instead of, the presence or absence of a label. Thus, the real time pattern that is generated may be quantitative (e.g., the number and sequence of light and dark signals) as well as qualitative (e.g., determining differences in a characteristic of the signals).

In an embodiment, either the polymerase or the primer comprises a label. In a particular embodiment, fluorescence resonance energy transfer (FRET) technology is employed to produce a detectable, and quenchable, label. Generally, a FRET donor (e.g., cyanine-3) is attached to the primer, to the polymerase, or to a previously incorporated nucleotide. The primer/template complex then is exposed to a nucleotide comprising a FRET acceptor (e.g., cyanine-5). If the nucleotide is incorporated, the acceptor is activated and emits detectable radiation, while the donor becomes dark. A detectable optical light signal indicates that a labeled nucleotide has been incorporated into the primer strand. In an embodiment, FRET may be used in the invention by modifying the primer to include a FRET donor molecule and using nucleotides labeled with a FRET acceptor molecule. The polymerase or previously incorporated nucleotides may also be modified to include a FRET donor molecule. In a particular embodiment, a polymerase is labeled with cyanine-3 and a nucleotide is labeled with cyanine-5.

The invention is useful in characterizing any form of nucleic acid, such as double-stranded DNA, single-stranded DNA, single-stranded DNA hairpins, DNA/RNA hybrids, RNAs with a recognition site for binding of the polymerizing agent, and RNA hairpins, for example. Characteristic temporal signal patterns of the invention are useful for comparing pairs or groups of nucleic acid templates.

Methods of the invention are useful for creating a unique identifier for a nucleic acid. Methods and temporal signal patterns of the invention are useful to compile information about a collection of nucleic acids and to create a unique identifier system, for example, for use in a clinical or research laboratory setting. Methods of the invention are also useful for screening and selecting nucleic acids of interest, for example, to undergo total sequencing, amplification, or cloning. Methods of the invention also comprise comparing two or more unique identifiers to determine if the nucleic acids comprise the same segment, have the same origin, are variants of one another, or are otherwise related.

The present invention also contemplates using a temporal signal pattern to identify known or novel gene mutations or polymorphisms. For example, methods of the invention are useful in characterizing genetic features such as, for example, VNTRs, STRs, microsatellite markers, SNPs, insertions, and deletions. For example, such methods may be used for identifying the presence of defective mismatch repair and genetic instability based on expansion of microsatellite repeat sequences in tumor DNA.

Medical applications of the invention include diagnosing diseases such as cancer, and screening for pre-cancerous disease states. The sensitivity of template-dependent polymerization reactions allows testing of a single cell or a few cells, which is useful, for example, in distinguishing between normal cells and cancerous or precancerous cells in a tumor biopsy. Methods of the invention may also be useful in clinical prenatal testing and diagnosis of fetal abnormalities. Because the invention is useful for characterizing single molecules, it can be adapted for use on a plurality of templates in an array format.

Methods of the invention are also useful in identifying a nucleic acid source, for example, identifying a species, organism, or individual of origin. The present invention is also useful for studying infectious diseases. For example, the invention provides an efficient method of characterizing isolated microorganisms and tracking the incidence, spread, and evolution of an infectious disease agent, as well as identifying novel microorganism or infectious agent variants. For example, in a viral epidemic, the present invention is useful in quickly diagnosing infected persons, characterizing viral strains, and cataloging new variants. In this respect, methods of the invention may be useful in the event of a bioterrorism attack. In addition to the infectious disease applications, the present invention is useful for identifying the origin of nucleic acid samples in forensic analysis, criminal investigations, and paternity or maternity testing.

The methods and compositions of the invention provide efficient analysis of a nucleic acid template because signal detection occurs at the time of nucleotide incorporation. Methods of the invention are simpler than whole-template sequencing and thus are particularly useful when determination of an entire sequence is not necessary.

### BRIEF DESCRIPTION OF THE DRAWINGS

The foregoing and other objects, features and advantages of the present invention, as well as the invention itself, will be more fully understood from the following description of preferred embodiments when read together with the accompanying drawings, in which:

FIG. 1A illustrates exemplary signals generated for four different templates on an array after a first nucleotide addition at Time (T)=1. A positive signal may comprise a characteristic such as light or color, for example. A negative signal may comprise a characteristic such as the absence of light or color, or may represent another color. In FIGS. 1A-1G, at least one nucleotide is unlabeled or comprises a label with a different characteristic (e.g., color) than the other nucleotides.

FIG. 1B illustrates an exemplary signal generated for four different templates on an array after the second nucleotide addition at T=2.

FIG. 1C illustrates an exemplary signal generated for four different templates on an array after the third nucleotide addition at T=3.

FIG. 1D illustrates an exemplary signal generated for four different templates on an array after the fourth nucleotide addition at T=4.

FIG. 1E illustrates an exemplary signal generated for four different templates on an array after the fifth nucleotide addition at T=5.

FIG. 1F illustrates an exemplary signal generated for four different templates on an array after the sixth nucleotide addition at T=6.

FIG. 1G represents the temporal pattern of signals generated for each of the four template nucleic acids shown in FIGS. 1A-1F, from T=1 to T=6.

FIG. 2 is a diagrammatical representation of total internal reflection fluorescence (TIRF) microscopy for two-dimensional imaging.

FIG. 3 illustrates a temporal signal pattern generated using FRET.

### DETAILED DESCRIPTION OF THE INVENTION

The invention provides methods and compositions for characterizing a nucleic acid. The invention provides methods and compositions for generating a signal pattern that uniquely identifies a nucleic acid. The invention comprises template dependent polymerization reactions in which fewer than all of the nucleotides necessary to obtain a full-sequence complement are labeled. Detecting a subset of nucleotide incorporations during template dependent polymerization generates a useful signature for a nucleic acid template.

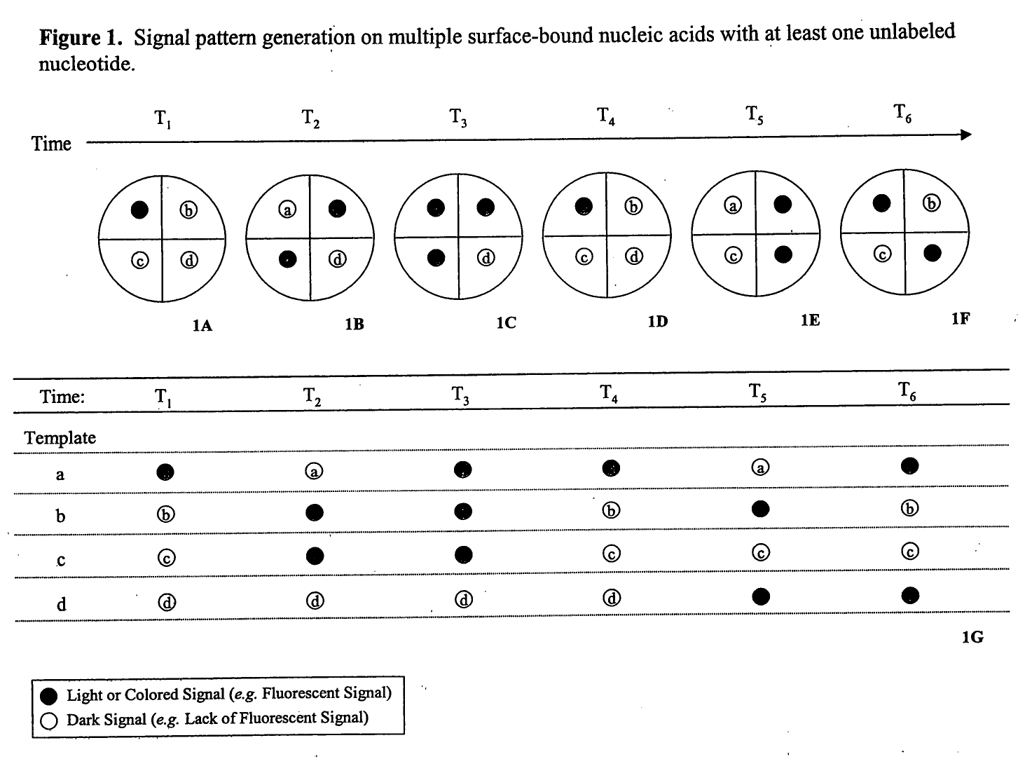

According to the invention, a template-dependent polymerization reaction is conducted on a nucleic acid of interest such that a signal pattern is created, which is unique to the template. FIGS. 1A through 1F illustrate the signals generated during template-dependent polymerization of six nucleotide additions on four templates. FIG. 1G illustrates a temporal signal pattern generated by primer-directed nucleotide polymerization using from 1 to 3 labeled nucleotides and at least one unlabeled or differently labeled nucleotide. In one embodiment, the reaction comprises a series of incorporation events, wherein fewer than all types of the nucleotide species present in a nucleic acid template (e.g., which is normally four: A, G, C, and T or U) comprise a detectable label. The incorporation of labeled nucleotides can be detected in real-time and the incorporation events can be monitored to generate a real time pattern. Real-time, or “movie mode,” detection of the polymerization reaction results in a pattern representing the presence or absence of signals, the resulting pattern representing a characteristic of the nucleic acid template.

Nucleic acid characterization according to the invention is an efficient method of creating a unique identifier for a template. Signal patterns generated according to the invention are useful for identifying individual nucleic acids, comparing two or more nucleic acids, or distinguishing a nucleic acid from other similar templates. Temporal signal patterns comprise compilations of positive or negative (e.g., light, dark, and/or color) detection events that are used to uniquely identify a nucleic acid. Detection events may also comprise non-light based signals, for example heat or radioactivity release. In one embodiment, methods of the invention contemplate translating the detection events into a representative signal pattern that is stored, for example, as a code, an illustration, or a binary series. The signal pattern may be compared to a signal pattern of another nucleic acid that was generated at a different time and also stored. In an alternative embodiment, methods of the invention contemplate generating signal patterns for a plurality of nucleic acids simultaneously, while the detection event patterns are compared in real-time.

In one embodiment, methods of the invention contemplate detecting an incorporation event as it occurs. In an embodiment, a nucleic acid is exposed to one labeled dNTP, where N=one of A, G, C, T, or U, in the presence of the other three unlabeled dNTPs. In some embodiments, a nucleic acid is exposed to two labeled and two unlabeled nucleotides, or three labeled nucleotides and one unlabeled nucleotide. Methods of the invention also contemplate exposing a template to all four labeled nucleotides, wherein each nucleotide has a distinct label. Rather than creating a signal pattern comprising, for example, light and dark temporal events, such an embodiment would generate, for example, a four-colored signal pattern indicative of a nucleic acid. In addition, if the template may only comprise at most two or three nucleotide species, then only one or two labeled nucleotides are used and one or two unlabeled or differently labeled nucleotides are used.

In some applications, the species of labeled nucleotide is immaterial to the practice or composition of the invention. For example, when comparing a plurality of unidentified templates in real time, as long as signal patterns are generated under the same reaction conditions, the comparison is independent of the specific labeled nucleotide species used. In other applications, however, it may be important to choose a particular labeled nucleotide species or to note which species was used to create a signal pattern, for example, when comparing nucleic acids that were not characterized simultaneously. Additionally, if the nucleic acid comprises a known region that has a particular sequence structure, it may be advantageous to choose a labeled species depending on certain genetic features, such as known polymorphisms, mutation hot spots, or G-C rich sequences, for example.

Methods according to the invention provide simple and accurate nucleic acid characterization, with applications in disease detection and diagnosis and individual and comparative genome analysis. Methods according to the invention provide DNA fingerprinting, polymorphism identification, for example single nucleotide polymorphism (SNP) detection, as well as applications in cancer diagnosis and therapeutic treatment selection. Applied to RNA sequences, methods according to the invention identify alternate splice sites, enumerate copy number, measure gene expression, identify unknown RNA molecules present in cells at low copy number, annotate genomes by determining which sequences are actually transcribed, determine phylogenic relationships, elucidate differentiation of cells, and facilitate tissue engineering. Methods according to the invention also are used to analyze activities of other biomacromolecules such as RNA translation and protein assembly.

Target Nucleic Acids

According to the invention, nucleic acid templates may be derived from a variety of sources. For example, nucleic acids may be naturally occurring DNA or RNA isolated from any source, recombinant molecules, cDNA, or synthetic analogs, as known in the art. The nucleic acid template may comprise genomic DNA, DNA fragments (e.g., exons, introns, regulatory elements, such as promoters, enhancers, initiation and termination regions, expression regulatory factors, expression controls, and other control regions), DNA comprising one or more single-nucleotide polymorphism (SNP), and allelic and mutant variants. The nucleic acid template may also be an RNA, such as mRNA, tRNA, rRNA, ribozymes, splice variants, antisense RNA, or RNAi. Also contemplated as useful according to the invention are RNA with a recognition site for binding a polymerase, transcripts of a single cell, organelle or microorganism, and all or portions of RNA complements of one or more cells, for example, cells from different stages of development, differentiation, or disease, and cells from different species.

Nucleic acids may be obtained from any nucleic acid source, such as a cell of a person, animal, insect, or plant, or cellular or microbial organism, such as a bacteria, or other infectious agent, such as a virus. Individual nucleic acids may be isolated for analysis, for example, from single cells in a patient sample comprised of cancerous and precancerous cells. Nucleic acids may be processed prior to depositing on a surface, for example, by restriction digestion, shearing, amplification, or cloning. In a preferred embodiment, a nucleic acid is genomic DNA extracted from one or more cells.

Many methods are available for the isolation and purification of nucleic acid templates for use in the present invention. Preferably, the target molecules or nucleic acids are sufficiently free of proteins and any other interfering substances to allow target-specific primer annealing and extension. Preferred purification methods include (i) organic extraction followed by ethanol precipitation, e.g., using a phenol/chloroform organic reagent, preferably using an automated DNA extractor, e.g., a Model 341 DNA Extractor available from PE Applied Biosystems (Foster City, Calif.); (ii) solid phase adsorption methods; and (iii) salt-induced nucleic acid precipitation methods, such methods being typically referred to as “salting-out” methods. Optimally, each of the above purification methods is preceded by an enzyme digestion step to help eliminate protein from the sample, e.g., digestion with proteinase K or other like protease.

Generally, nucleic acid templates may have a length of about 5 bases, about 10 bases, about 20 bases, about 30 bases, about 40 bases, about 50 bases, about 60 bases, about 70 bases, about 80 bases, about 90 bases, about 100 bases, about 200 bases, about 500 bases, about 1 kb, about 3 kb, about 10 kb, or about 20 kb and so on. Preferably, nucleic acid templates are about 10 to about 50 bases.

Depending on the template, a DNA polymerase, an RNA polymerase, a reverse transcriptase, or any enzyme capable of polymerizing a nucleic acid strand complementary to the nucleic acid template may be used in the primer extension reactions. Generally, the polymerase according to the invention has high incorporation accuracy and a processivity (number of nucleotides incorporated before the polymerase dissociates from the target nucleic acid) of at least about 20 nucleotides. Nucleotides may be selected to be compatible with the polymerase.

Nucleotides

Nucleotides useful in the invention include both naturally-occurring and modified or non-naturally occurring nucleotides, and include nucleotide analogues. A nucleotide according to the invention may be, for example, a ribonucleotide, a deoxyribonucleotide, a dideoxyribonucleotide, a modified ribonucleotide, a modified deoxyribonucleotide, a peptide nucleotide, a modified peptide nucleotide or a nucleotide having a modified phosphate-sugar backbone.

Labeled nucleotides of the invention include any nucleotide that has been modified to include a label that is directly or indirectly detectable. Such labels include optically-detectable labels such as fluorescent labels, including fluorescein, rhodamine, derivatized rhodamine dyes, such as TAMRA, phosphor, polymethadine dye, fluorescent phosphoramidite, texas red, green fluorescent protein, acridine, cyanine, cyanine-5 dye, cyanine-3 dye, 5-(2′-aminoethyl)-aminonaphthalene-1-sulfonic acid (EDANS), BODIPY, ALEXA, or a derivative or modification of any of the foregoing. In an embodiment, a nucleotide labeled with a fluorescent dye is directly detected after incorporation into a primer and subsequent wash step that removes unincorporated nucleotides. In another embodiment of the invention, fluorescence resonance energy transfer (FRET) technology is employed to produce a detectable, and quenchable, label. While the invention is exemplified herein with fluorescent labels, the invention is not so limited and may be practiced using nucleotides labeled with any form of detectable label, including radioactive labels, chemoluminescent labels, luminescent labels, phosphorescent labels, fluorescence polarization labels, and charge labels, for example.

Characterization methods according to the invention include exposing a nucleic acid template to at least one nucleotide, labeled nucleotide, or nucleotide analog allowing for extension of the primer. A nucleotide or nucleotide analog includes any base or base-type including adenine, cytosine, guanine, uracil, or thymine bases. Additional nucleotide analogs include xanthine or hypoxanthine, 5-bromouracil, 2-aminopurine, deoxyinosine, or methylated cytosine, such as 5-methylcytosine, N4-methoxydeoxycytosine, and the like. Also included are bases of polynucleotide mimetics, such as methylated nucleic acids, e.g., 2′-O-methRNA, peptide nucleic acids, modified peptide nucleic acids, and any other structural moiety that acts substantially like a nucleotide or base, for example, by exhibiting base-complementarity with one or more bases that occur in DNA or RNA and/or being capable of base-complementary incorporation.

According to the invention, identification of nucleotide incorporation events may be accomplished using fluorescence resonance energy transfer (FRET). FRET confers an advantage of detecting signal over background fluorescence. Generally, a FRET donor (e.g., cyanine-3) is placed on the primer, on the polymerase, or on a previously incorporated nucleotide. The primer/template complex then is exposed to a nucleotide comprising a FRET acceptor (e.g., cyanine-5). If the nucleotide is incorporated, the acceptor is activated and emits detectable radiation, while the donor goes dark. In one embodiment, a dye-labeled polymerase is provided, wherein the dye label is able to transfer energy to the label on a dNTP upon incorporation into the primer according to Watson-Crick base pairing rules.

The fluorescently labeled nucleotides may be obtained commercially (e.g., from Amersham Biosciences, Piscataway, N.J.). Alternatively, fluorescently labeled nucleotides may also be produced by various techniques, such as those described in Kambara et al. (1988) Bio/Technol., 6:816-21; Smith et al. (1985) Nucl. Acid Res., 13: 2399-2412; and Smith et al.(1986) Nature, 321: 674-79. The fluorescent dye is preferably linked to the deoxyribose by a linker arm that is easily cleaved by chemical or enzymatic means. The length of the linker between the dye and the nucleotide can impact the incorporation rate and efficiency. See Zhu et al. (1997) Cytometry, 28: 206. There are numerous linkers and methods for attaching labels to nucleotides, as shown in Oligonucleotides and Analogues: A Practical Approach (1991) (IRL Press, Oxford); Zuckerman et al. (1987) Polynucleotides Res., 15: 5305-21; Sharma et al. (1991) Polynucleotides Res., 19: 3019; Giusti et al. (1993) PCR Methods and Applications, 2: 223-27; Fung et al., U.S. Pat. No. 4,757,141; Stabinsky, U.S. Pat. No. 4,739,044; Agrawal et al. (1990) Tetrahedron Letters, 31: 1543-46; Sproat et al. (1987), Polynucleotides Res., 15: 4837; and Nelson et al. (1989) Polynucleotides Res., 17: 7187-94. Extensive guidance exists in the literature for derivatizing fluorophore and quencher molecules for covalent attachment via common reactive groups that may be added to a nucleotide. Many linking moieties and methods for attaching fluorophore moieties to nucleotides also exist, as described in Oligonucleotides and Analogues, supra; Guisti et al., supra; Agrawal et al, supra; and Sproat et al., supra.

Removal of Blocking Group and Labels

After detecting a fluorescent signal, it may be desirable to eliminate residual fluorescence or allow it to disappear in order to recognize dark signaling events of the invention. A fluorophore may self-extinguish after emission. Alternatively, a bleaching or cleaving step may be performed to remove remaining fluorescence.

After detecting incorporation of a labeled nucleotide, the label may be removed before repeating the polymerization process to generate a light, dark, or color signal at the next locus on the template. Removal of the label can be effected by removal of the labeled nucleotide using a 3′-5′ exonuclease and subsequent replacement with an unlabeled nucleotide. Preferably, however, the label is attached to the nucleotide by a cleavable linker such that the label is easily removed after detection. Disulfide linkers, for example, are well-known in the art and are easily cleaved using a thiol reagent. In a further alternative, where the label is a fluorescent label, it is possible to neutralize the label by bleaching it with radiation. Photobleaching can be performed according to methods, e.g., as described in Jacobson et al. (1973) “International Workshop on the Application of Fluorescence Photobleaching Techniques to Problems in Cell Biology,” Federation Proceedings, 42: 72-79; Okabe et al. (1993) J. Cell Biol. 120: 1177-86; and Close et al. (1973) Radiat. Res. 53: 349-57.

Methods of the invention also contemplate non-labeled nucleotide procedures. For example, nucleotide incorporation may be monitored by detection of pyrophosphate release (see, e.g., WO98/13523, WO98/28440, and Ronaghi et al. (1998) Science 281:363). A pyrophosphate-detection enzyme cascade may be included in the reaction mixture in order to produce a chemoluminescent signal. Alternatively, instead of deoxynucleotides or dideoxynucleotides, nucleotide analogues may be used, which are capable of acting as substrates for the polymerase but incapable of acting as substrates for the pyrophosphate-detection enzyme. Pyrophosphate is released upon incorporation of a deoxynucleotide or dideoxynucleotide, which can be detected enzymatically. This method employs no wash steps, relying instead on continual addition of reagents. In an embodiment, addition of a nucleotide analog, rather than a labeled nucleotide, generates a signal pattern embodied in dark events, which is analogous to a light event signal pattern generated by a labeled nucleotide.

Primers

If the sequence of part of the region downstream of the nucleic acid segment to be analyzed is known, a specific primer may be constructed and hybridized to this region of the nucleic acid template. Alternatively, if sequences of the downstream region on the nucleic acid template are not known, universal, degenerate, or random primers may be used in various primer combinations. Alternatively, known sequences may be biotinylated and ligated to the target nucleic acids. In yet another approach, a nucleic acid may be digested with a restriction endonuclease, and primers designed to hybridize with the known restriction sites that define the ends of the fragments produced.

Extension primers may be synthetically made using conventional nucleic acid synthesis techniques. For example, primers may be synthesized on an automated DNA synthesizer, e.g. an Applied Biosystems, Inc. model 392 or 394 DNA/RNA Synthesizer (Foster City, Calif.), using standard chemistries, such as phosphoramidite chemistry, and the like. Alternative chemistries, e.g., resulting in non-natural backbone groups, such as phosphorothioate and the like, may also be employed provided that, for example, the resulting oligonucleotides are compatible with the polymerizing agent. The primers may also be ordered commercially from a variety of companies that specialize in custom nucleic acids such as Operon Inc. (Alameda, Calif.).

In some instances, the extension primer includes a label. When hybridized to a surface-linked nucleic acid molecule, the label facilitates locating the bound molecule through imaging. For example, the primer is labeled with a fluorescent labeling moiety (e.g., Cy3 or Cy5), or any other means used to label nucleotides. The detectable label on the primer may be different from the label on the nucleotides or nucleotide analogs in the subsequent extension reactions. In an embodiment of the invention, after locating a support-bound template, the primer label is extinguished by photobleaching or cleaving the fluorophore as described above. Suitable fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodarnine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolla Blue; phthalo cyanine; and naphthalo cyanine.

The primer may be hybridized to a template before or after the template is attached on a surface of a substrate or array. Primer annealing is performed under conditions that are stringent enough to require sufficient sequence specificity, yet permissive enough to allow formation of stable hybrids at an acceptable rate. The temperature and time required for primer annealing depend upon several factors including nucleotide composition, nucleic acid length, and the concentration of the primer; the nature of the solvent used, for example, the concentration of dimethylsulfoxide (DMSO), polyethylene glycol (PEG), formamide, or glycerol; as well as the concentrations of counter ions, such as magnesium and manganese. Typically, hybridization with synthetic polynucleotides is carried out at a temperature that is about 5° C. to about 10° C. below the melting temperature (Tm) of the target polynucleotide-primer complex in the annealing solvent.

After attaching a nucleic acid template to a surface, template-dependent primer extension reactions may be performed to analyze the temporal signal pattern of the nucleic acid template. The primer is extended by a polymerase in the presence of a labeled or unlabeled nucleotide or nucleotide analog or mixture thereof at a temperature of about 10° C. to about 70° C., about 20° C. to about 60° C., about 30° C. to about 50° C., or preferably at about 37° C.

Surface and Anchoring

A target nucleic acid may be immobilized or anchored on a substrate to prevent its release into surrounding solution or other medium. For example, a primer, a nucleic acid template, polymerase, or a primer/template complex may be anchored or immobilized by covalent bonding, non-covalent bonding, ionic bonding, hydrogen bonding, van der Waals forces, hydrophobic bonding, or a combination thereof.

In some embodiments, target nucleic acids are attached to a substrate for signal pattern characterization. In these embodiments, the nucleic acid may be attached to the substrate through a covalent linkage or a non-covalent linkage. When the nucleic acid is attached to the substrate through a non-covalent linkage, the nucleic acid includes one member of specific binding pair, e.g., biotin, the other member of the pair being attached to the substrate, e.g., avidin or streptavidin. Other useful binding pairs include, for example, an antigen-antibody binding pair, photoactivated coupling molecules, digoxigenin/anti-digoxigenin, and a pair of complementary nucleic acids. Several methods are available for covalently linking polynucleotides to substrates, e.g., through reaction of a 5′-amino polynucleotide with an isothiocyanate-functionalized glass support. A wide range of exemplary linking moieties for attaching primers onto solid supports, either covalently or non-covalently, are known in the art.

Methods of the invention comprise conducting primer extension reactions with target nucleic acids that are attached to a substrate, surface, support, or an array. For example, each member of the plurality of target nucleic acids may be covalently attached to a surface that has reduced background fluorescence with respect to glass, polished glass, fused silica, or plastic. Examples of surfaces appropriate for the invention include, for example, polytetrafluoroethylene or a derivative of polytetrafluoroethylene, such as silanized polytetrafluoroethylene, polytetrafluoroethylene, epoxides, derivatized epoxides, polyelectrolyte multilayers, and others.

The surface to which oligonucleotides are attached may be chemically modified to promote attachment, improve spatial resolution, and/or reduce background. Exemplary substrate coatings include polyelectrolyte multilayers. Typically, these are made via alternate coatings with positive charge (e.g., polyallylamine) and negative charge (e.g., polyacrylic acid). Alternatively, the surface may be covalently modified, as with vapor phase coatings using 3-aminopropyltrimethoxysilane. In one embodiment, the primer attaches to the solid support by direct amine end attachment of the 3′ end of primer. In another embodiment, a fused silica surface is modified with alternating layers of pollyallylamine and polyacrylic acid. The final layer comprises polyacrylic acid, which is then used to bind a biotin layer, followed by a streptavidin layer. Biotin modified nucleic acids are then attached to the surface via streptavidin-biotin linkage.

Solid supports of the invention may comprise glass, fused silica, epoxy, plastic, metal, nylon, gel matrix or composites. Furthermore, the substrate or support may include a semi-solid support (e.g., a gel or other matrix), and/or a porous support (e.g., a nylon membrane or other membrane). In an embodiment, the surface of the solid support is coated with epoxide. The surface of the substrate or support may be planar, curved, pointed, or any suitable two-dimensional or three-dimensional geometry. The invention also contemplates the use of beads or other non-fixed surfaces, as well as commercially-available or custom-made arrays. Target molecules or nucleic acids may be synthesized on a substrate to form a substrate including regions coated with nucleic acids or primers, for example. In some embodiments, the substrate is uniformly comprised of nucleic acid targets or primers. That is, within each region in a substrate or array, the same nucleic acid or primer may be synthesized.

Detection & Equipment

Any detection method may be used that is suitable for the type of label employed. Thus, exemplary detection methods include optical emission detection, such as fluorescence or chemiluminescence detection, radioactive detection, and optical absorbance detection, such as UV-visible absorbance detection. For example, nucleotide incorporation patterns may be detected on a substrate by scanning all or portions of each substrate simultaneously or serially, depending on the scanning method used. For fluorescence labeling, selected regions on a substrate may be serially scanned one-by-one or row-by-row using a fluorescence microscope. Hybridization patterns may also be scanned using a CCD camera (e.g., Model TE/CCD512SF, Princeton Instruments, Trenton, N.J.) with suitable optics, such as described total internal reflection optics, or may be imaged by TV monitoring. To detect radioactive signals, a phosphorimager device may be used. Other commercial suppliers of imaging instruments include General Scanning Inc., (Watertown, Mass.), Genix Technologies (Waterloo, Ontario, Canada), and Applied Precision LLC (Issaquah, Wash.). Such detection methods are particularly useful to achieve simultaneous scanning of multiple tag complement regions. As such, some embodiments of the present invention provide for detection of a single nucleotide into a single target nucleic acid molecule. A number of methods are available for this purpose. Methods for visualizing single molecules within nucleic acids labeled with an intercalating dye include, for example, fluorescence microscopy. For example, the fluorescent spectrum and lifetime of a single molecule excited-state can be measured. Standard detectors such as a photomultiplier tube or avalanche photodiode may be used. Full field imaging with a two-stage image intensified COD camera also may be used. Additionally, low noise cooled CCD may also be used to detect single fluorescent molecules.

The detection system for the signal may depend upon the labeling moiety used, which is defined by the chemistry available. For optical signals, a combination of an optical fiber or charged couple device (CCD) may be used in the detection step. In those circumstances where the substrate is itself transparent to the radiation used, it is possible to have an incident light beam pass through the substrate with the detector located opposite the substrate from the target nucleic acid. For electromagnetic labeling moieties, various forms of spectroscopy systems may be used. Various physical orientations for the detection system are available and discussion of important design parameters is provided in the art.

A number of approaches may be used to detect incorporation of fluorescently-labeled nucleotides into nucleic acid templates. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. It is sometimes referred to as a high-efficiency photon detection system. Suitable photon detection systems include, but are not limited to, photodiodes and intensified CCD cameras. For example, an intensified charge couple device (ICCD) camera may be used. The use of an ICCD camera to image individual fluorescent dye molecules in a fluid near a surface provides numerous advantages. For example, with an ICCD optical setup, it is possible to acquire a series of images (movies) of fluorophores.

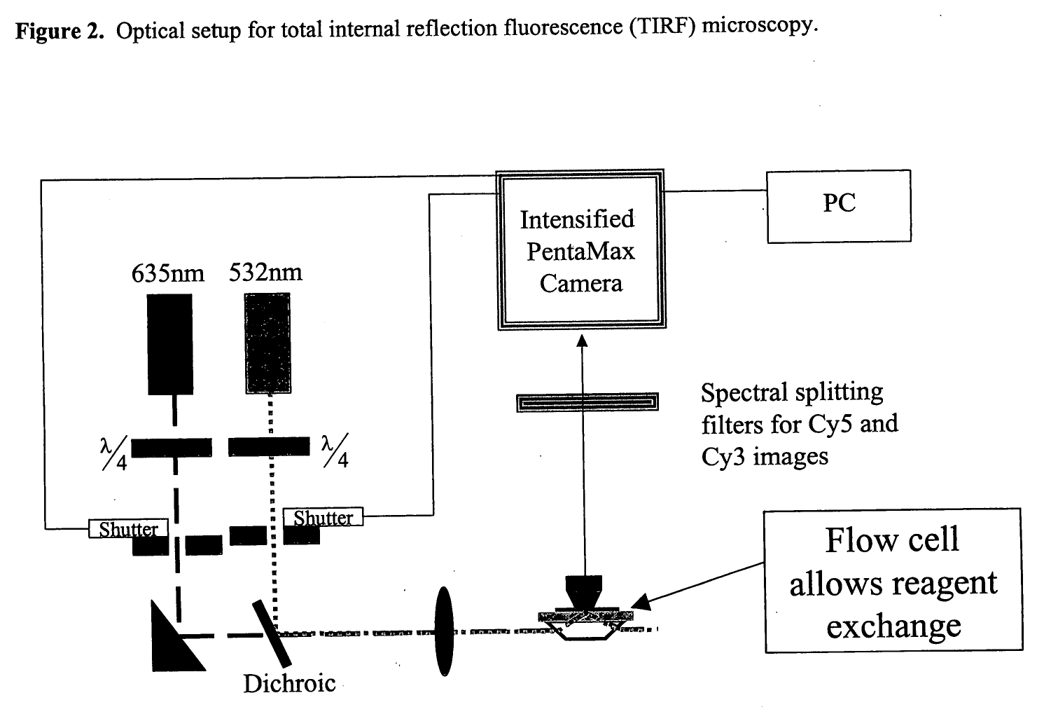

Some embodiments of the present invention use total internal reflection fluorescence (TIRF) microscopy for two-dimensional imaging, as shown in FIG. 2. Total internal reflection microscopy uses totally internally reflected excitation light and is well known in the art. In certain embodiments, detection is carried out using evanescent wave illumination and total internal reflection fluorescence microscopy. An evanescent light field may be set up at the surface, for example, to image fluorescently-labeled polynucleotide molecules. When a laser beam is totally reflected at the interface between a liquid and a solid substrate (e.g., a glass slide), the excitation light beam penetrates only a short distance into the liquid. In other words, the optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the immobilized nucleic acid template-primer complex in the presence of a polymerase. TIRF microscopy may then be used to visualize the immobilized nucleic acid template-primer complex and/or the incorporated nucleotides with single molecule resolution. With TIRF technology, the excitation light (e.g., a laser beam) illuminates only a small volume of solution close to the substrate, called the excitation zone. Signals from free (i.e., unincorporated) nucleotides in solution outside the excitation zone would not be detected. Signals from free nucleotides that diffuse into the excitation zone would appear as a broad band background because the free nucleotides move quickly across the excitation zone.

Measured signals may be analyzed manually or by appropriate computer methods to tabulate results. The substrates and reaction conditions may include appropriate controls for verifying the integrity of hybridization and extension conditions, and for providing standard curves for quantification, if desired. For example, a control primer may be added to the polynucleotide sample for extending a target nucleic acid sequence that is known to be present in the sample or a target nucleic acid sequence that is added to the sample. The absence of the expected extension product is an indication that there is a defect with the sample or assay components requiring correction.

Methods of the invention also contemplate bulk nucleic acid techniques. Although not necessary for the practice of the invention, template amplification and bulk detection may be used with the methods described. Detection of signal pattern representing incorporation events may comprise, for example, optical recognition of fluorescent activity in a microtiter plate, eppendorf tube, or capillary tube.

In an embodiment, control templates are synthesized and included in the reaction chamber to demonstrate successful light, dark, and/or color signal pattern generation. Control templates may comprise, for example, four variations of a four base repeat (e.g., GATC), such that only one template will have a detectable light incorporation event at a time, when conducting an experiment with only one labeled nucleotide species. In effect, when using one labeled nucleotide and three unlabeled nucleotide species, the labeled nucleotide will be incorporated at locus 1 of template 1, locus 2 of template 2, locus 3 of template 3, locus 4 of template 4, locus 5 of template 1, locus 6 of template 2, locus 7 of template 3, and so on. Unlabeled nucleotides will fill in, as with the template of interest. A set of exemplary control templates (T) includes: T1, (GATC)n; T2, (CGAT)n; T3 (TCGA)n; T4, (ATCG)n. Thus, if using labeled dGTP, incorporation events will be detectable on one template at a time and on each template every fourth polymerization cycle.

Practice of the invention will be still more fully understood from the following examples, which are presented herein for illustration only and should not be construed as limiting the invention in any way.

### EXEMPLIFICATION

### Example 1

### Generating a Signal Pattern

In this example, a temporal signal pattern is generated for a DNA template by detecting incorporation of dUTP-Cy5. A nucleic acid template is obtained from a cell or tissue, for example, using one of a variety of procedures for extracting nucleic acids, which are well known in the art. While the invention is exemplified below with synthetic oligonucleotides, the invention is not so limited and may be practiced using any nucleic acid template, including genomic DNA, cDNA, such as cDNA library, and RNA.

The template is 5′-biotinylated and is hybridized to a primer. A surface is chemically modified with polyelectrolytes to minimize fluorescence background from non-specific binding of free nucleotides and other debris. The surface is further prepared with streptavidin to facilitate binding of the biotinylated template. A solid support comprising reaction chambers having a fused silica surface is sonicated in 2% MICRO-90 soap (Cole-Parmer, Vernon Hills, Ill.) for 20 minutes and then cleaned by immersion in boiling RCA solution (6:4:1 high-purity H₂O/30% NH₄OH/30% H₂O₂) for 1 hour. The solid support is then immersed alternately in positively charged polyallylamine (Aldrich Chemical, Milwaukee, Wis.) and negatively charged polyacrylic acid (Aldrich Chemical) at 2 mg/ml and pH 8.0 for 10 minutes each and washed intensively with distilled water between immersions. The slides are incubated with 5 mM biotin-amine reagent (Biotin-EZ-Link, Pierce, Rockford, Ill.) for 10 minutes in the presence of 1-[3-(dimethylamino)propyl]-3-ethylcarbodiimide hydrochloride (EDC, Sigma-Aldrich, St. Louis, Mo.) in MES buffer, followed by incubation with Streptavidin Plus (Prozyme, San Leandro, Calif.) at 0.1 mg/ml for 15 minutes in Tris buffer. The biotinylated single-stranded template is deposited onto the streptavidin-coated chamber surface at 10 pM for 10 minutes in Tris buffer that contains 100 mM MgCl₂.

Fluorescence detection is performed with an upright microscope (BH-2, Olympus, Melville, N.Y.) equipped with total internal reflection (TIR) illumination, such as the BH-2 microscope from Olympus (Melville, N.Y.). Two laser beams, 635 (Coherent, Santa Clara, Calif.) and 532 nm (Brimrose, Baltimore, Md.), with nominal powers of 8 and 10 mW, respectively, are circularly polarized by quarter-wave plates and undergo TIR in a dove prism (Edmund Scientific, Barrington, N.J.). The prism is optically coupled to the fused silica bottom (Esco, Oak Ridge, N.J.) of the reaction chambers so that evanescent waves illuminated up to 150 nm above the surface of the fused silica. An objective (DPlanApo, 100 UV 1.3oil, Olympus, Melville, N.Y.) collects the fluorescence signal through the top plastic cover of the chamber, which is deflected by the objective to approximately 40 μm from the silica surface. An image splitter (Optical Insights, Santa Fe, N. Mex.) directs the light through two bandpass filters (630dcxr, HQ585/80, HQ690/60; Chroma Technology, Brattleboro, Vt.) to an intensified charge-coupled device (I-PentaMAX; Roper Scientific, Trenton, N.J.), which records adjacent images of a 120×60 μm section of the surface in two colors.

Experimental Protocols

FRET-Based Method Using Nucleotide-Based Donor Fluorophore

In a first experiment, a primer is hybridized to a complementary sequence present in the support-bound DNA template. A first donor molecule (e.g., cyanine-3), which is attached to a nucleotide, is introduced into the extending primer sequence. Further nucleotides comprising unlabeled dNTPs and acceptor fluorophores (e.g., cyanine-5) are added in a template-dependent manner. In general, each cycle of nucleotide addition preferably is regulated to allow incorporation of about 1 base. An incorporation event comprises either a light signal, a dark signal, or a different color signal or a combination thereof, indicating incorporation of a labeled or an unlabeled nucleotide.

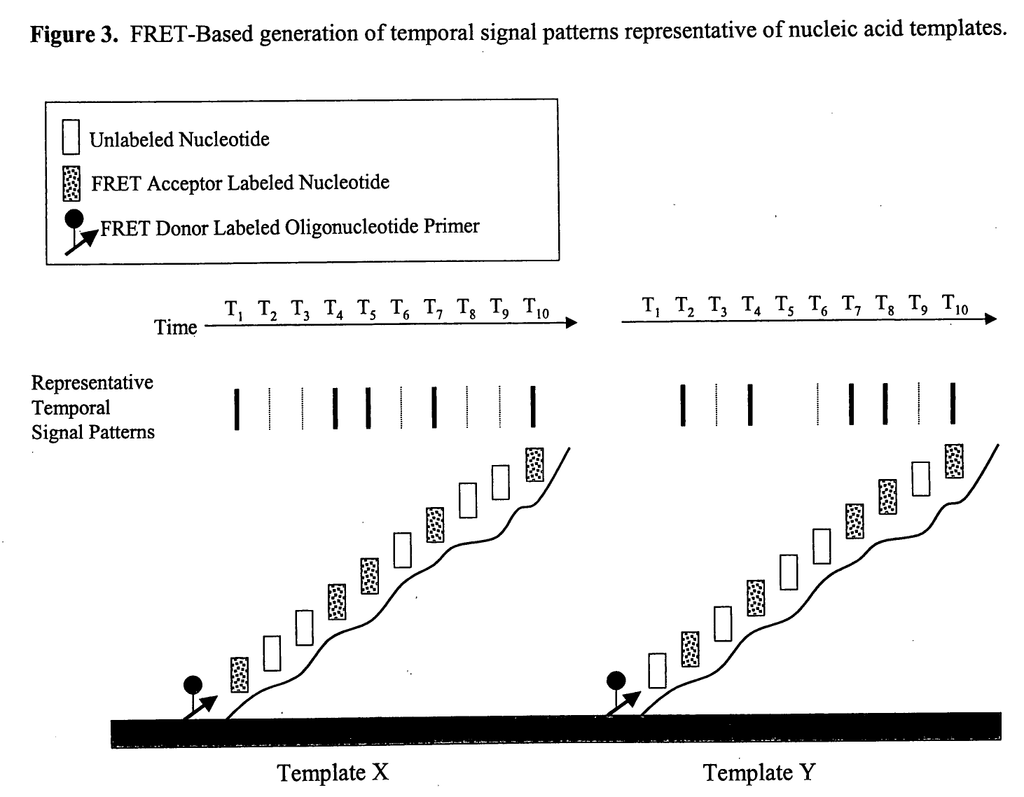

The Förster radius of Cy3/Cy5 fluorophore pairs is about 5 nm (or about 15 nucleotides, on average). Thus, a donor molecule must be reintroduced into the growing strand approximately every 15 bases, if the desired length of the signal pattern exceeds about 15. Where, as here, fewer than all four dNTPs comprise an acceptor molecule, a non-acceptor dNTP may be used as a donor. In this example, dUTP-Cy5 is the acceptor, therefore, any of dGTP, dCTP, or dATP may comprise the donor. FIG. 3 shows schematically the process of FRET-based, template-dependent nucleotide addition as described in this example. In some FRET-based embodiments, a signal pattern of the invention is representative of about 2 to about 16 nucleotides, or any length in between.

The methods described above are alternatively conducted with the FRET donor attached to the polymerase molecule. In an embodiment, a donor-labeled polymerase follows the extending primer as new nucleotides bearing acceptor fluorophores are added. Thus, there typically is no requirement to refresh the donor. In another embodiment, the same methods are carried out using a nucleotide binding protein (e.g., DNA binding protein) as the carrier of a donor fluorophore. In that embodiment, the DNA binding protein is spaced at intervals (e.g., about 5 nm or less) to allow FRET. Thus, there are many alternatives for using FRET to conduct single molecule characterization with respect to methods and compositions of the invention. It is not required, however, that FRET be used as the detection method. Rather, because of the intensities of the FRET signal with respect to background, FRET is a useful alternative when background radiation is relatively high.

Non-FRET Based Methods

As noted above, any source of DNA or RNA is appropriate for use in methods of the invention. Typically, a small (e.g., 25 ug) amount of DNA is digested to an average fragment size of 40 bp with 0.1 U DNase I (New England Biolabs) for 10 minutes at 37° C. Digested DNA fragment size is estimated by running an aliquot on a precast denaturing (TBE-Urea) 10% polyacrylamined gel (Novagen) and staining with SYBR Gold (Invitrogen/Molecular Probes). The DNase I-digested DNA is filtered through a YM10 ultrafiltration spin column (Millipore) to remove small digestion products (less than about 30 bp). Approximately 20 pmol of the filtered DNase I-digested product is polyadenylated with terminal transferase according to known methods (see, e.g., Roychoudhury, et al., Meth. Enzymol. 65(1): 43-62 (1980)). The resulting average dA tail length is 50±5 nucleotides. Terminal transferase is also used to label the same fragments with Cy3-dUTP. Fragments are then terminated with dideoxy TTP (also added by terminal transferase). The resulting fragments are again filtered in a YM10 ultrafiltration spin column and stored in water at −20° C.

Epoxide-coated glass slides (40 mm diameter #1.5 glass cover slips) (Erie Scientific, Portsmouth, N.H.) are preconditioned by soaking in 3×SSC for 15 minutes at 37° C. A 500 pM aliquot of 5′ aminated polydT(50) is incubated with each slide for 30 minutes at room temperature in a volume of 80 ml. The slides are then treated with phosphate (1M) for 4 hours at room temperature in order to passivate the surface. Slides, which contain a coating of polydT primer, are stored in rinse buffer (20 mM Tris, 100 mM NaCl, 0.001% Triton X-100, pH 8.0).

The slides are placed in a modified FCS2 flow cell (Bioptechs, Butler, Pa.) using a 50 um thick gasket. The flow cell is placed on a movable stage that is part of a high-efficiency fluorescence imaging system built around a Nikon TE 2000 inverted microscope equipped with a total internal reflection objective. The slide is rinsed with HEPES buffer containing 100 mM NaCl and equilibrated to 50° C. An aliqot of the fragments prepared as described above is diluted in 3×SSC to a final concentration of 1.2 nM, and a 100 ul aliquot is placed on the flow cell and incubated for 15 minutes. After incubation, the flow cell is rinsed with 1×SSC/HEPES/0.1% SDS followed by HEPES/NaCl. A passive vacuum is used to pull sample across the flow cell. The resulting slide contained duplex comprising the fragments described above hybridized to polydT capture primers. The temperature of the flow cell was then reduced to 37° C.

For sequencing, CTP, GTP, ATC, and UTP, each having a cyanine-5 label (on the 7-deaza position for ATP and GTP and at the c5 position for CTP and UTP) were sequentially presented in the flow cell. Initial imaging is used to determine the location of duplex in the flow cell. A 532 nm laser (Verdi V-2, Coherent, Santa Calara, Calif.) is used to image the Cy3 label attached to duplex. Once the location of duplex is noted and recorded, Cy5 labeled nucleotides are presented. Nucleotide imaging is conducted using a 635 nm laser. First 5 uM dCTP-Cy5 is placed in the flow cell for 5 minutes. It is incorporated into any primer in which there is complementary binding with the template base opposite the 3′ end of the primer. After incubation, the slide is rinsed in 1×SSC/15 mM HEPES/0.1% SDS/pH 7.0 (“SSC/HEPES/SDS”) 15 times at 60 ul volumes each. That rinse is followed by a rinse with 150 mM HEPES/150 mM NaCl/pH 7.0 (“HEPES/NaCl”), 10 times in 60 ul volumes each. An oxygen scavenger is added that contains 30% acetonitrile in 134 ul HEPES/NaCl, 24 ul 100 mM Tolox in MES pH 6.1, 10 ul DABCO in MES pH 6.1 8 ul 2M glucose, 20 ul NaI (50 mM stock in water), and 4 ul glucose oxidase. The slide is imaged (500 frames) for 0.2 seconds using an Inova301K laser at 647 nm, followed by imaging at 532 nm for 2 seconds to confirm duplex position. The positions having Cy-3 and Cy5 fluorescence are recorded. The flow cell is then rinsed 5 times each with SSC/HEPES/SDS (60 ul) and HEPES/NaCl (60 ul). Next label is cleaved using 50 mM TCEP for 5 minutes followed by a 5× rinse with SSC/HEPES/SDS (60 ul) and HEPES/NaCl (60 ul). The remaining nucleotide is then capped with 50 mM iodoacetamide followed by 5× rinsing with SSC/HEPES/SDS (60 ul) and HEPES/NaCl (60 ul). Scavenger is then reapplied and rinsed, and the other three nucleotides are presented in the manner described above for dCTP.

The procedure described above is repeated for a predetermined number of cycles until, for each attached duplex, a pattern of fluorescence image “stacks” is determined.

Software is employed to analyze the locations and intensities of fluorescence objects in the intensified charge-coupled device pictures. Fluorescent images acquired in the WinView32 interface (Roper Scientific, Princeton, N.J.) are analyzed using ImagePro Plus software (Media Cybernetics, Silver Springs, Md.). Essentially, the software is programmed to perform spot-finding in a predefined image field using user-defined size and intensity filters. The program then assigns grid coordinates to each identified spot, and normalizes the intensity of spot fluorescence with respect to background across multiple image frames. From those data, specific incorporated nucleotides are identified. Generally, the type of image analysis software employed to analyze fluorescent images is immaterial as long as it is capable of being programmed to discriminate a desired signal over background. The programming of commercial software packages for specific image analysis tasks is known to those of ordinary skill in the art.

In order to determine a signal pattern of the invention, the above protocol is performed sequentially in the presence of one species of labeled nucleotide and three species of unlabeled nucleotide. A signal pattern is compiled that is based upon serial incorporation of nucleotides into the extended primer. Multiple nucleic acid templates may be characterized on a single surface. Conducting a multiple template reaction may comprise a comparison of templates in individual fields. Alternatively, a multiple template reaction may be performed to characterize unrelated templates.

### Incorporation by Reference

The contents of all cited references (including literature references, patents, and patent applications) that may be cited throughout this application are hereby expressly incorporated by reference. The practice of the present invention will employ, unless otherwise indicated, conventional techniques of nucleic acid preparation, manipulation, and sequencing, which are well known in the art.

### Equivalents

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

We claim:

1. A temporal signal pattern that represents a characteristic of a nucleic acid template, the signal pattern comprising signals that are generated by incorporation events in a template-dependent nucleic acid polymerization reaction, wherein at least one, but fewer than four, different nucleotide triphosphates present in the reaction comprises an optically-detectable label, and wherein presence or absence of the label is sequentially detected on the template, thereby generating a temporal signal pattern that represents a characteristic of the template.

2. The pattern according to claim 1, wherein primer/template duplex is attached to a surface.

3. The pattern according to claim 1, wherein primer/template duplex is attached to a surface such that the duplex is individually optically resolvable.

4. The pattern according to claim 1, wherein the characteristic is selected from the group consisting of a mutation and a polymorphism.

5. The pattern according to claim 1, wherein the characteristic represents a difference between the nucleotide sequence of two nucleic acid molecules.

6. The pattern according to claim 1, wherein the nucleic acid molecule is selected from the group consisting of genomic DNA, cDNA, and RNA.

7. The pattern according to claim 1, wherein the nucleic acid molecule is derived from a source selected from the group consisting of an animal tissue, an animal fluid, a plant tissue, a plant fluid, and a microorganism.

8. The pattern according to claim 1, wherein the label is a fluorescent label.

9. The pattern according to claim 1, wherein the label is a FRET acceptor.

10. The pattern according to claim 1, wherein the primer comprises a FRET donor.

11. The pattern according to claim 1, wherein the polymerase comprises a FRET donor.

12. The pattern according to claim 1, wherein the primer is labeled with cyanine-3 and the nucleotide is labeled with cyanine-5.

13. The pattern according to claim 1, wherein the polymerase is labeled with cyanine-3 and the nucleotide is labeled with cyanine-5.

14. The pattern according to claim 1, wherein the primer comprises an oligonucleotide.

15. The pattern according to claim 1, wherein the primer comprises a plurality of degenerate oligonucleotides.

16. The pattern according to claim 1, wherein the label is detected by a microscope having a total internal reflection objective.

17. The pattern according to claim 1, wherein the presence of the label is detected in real time.

18. The pattern according to claim 1, wherein the label is detected in real time until a signal pattern representative of 2, 3, 4, 5, 6, 7, 8, 9, 10, 11, 12, 13, 14, 15, or 16 nucleotides is determined.

19. The pattern according to claim 1, wherein the absence of the label is detected in real time.

20. The pattern according to claim 1, wherein the different nucleotide triphosphates comprise different labels.

21. The pattern according to claim 1, wherein the temporal signal pattern comprises at least two labels that are a different color.

22. A method for detecting a temporal signal pattern in a nucleic acid molecule, the method comprising the steps of:
(a) conducting a plurality of template-dependent nucleotide addition reactions in which a nucleotide triphosphate may be added to a primer that is hybridized to a nucleic acid template, wherein, at least one but less than four different types of labeled nucleotide triphosphates comprises an optically-detectable label; and
(b) detecting a temporal pattern of addition of each nucleotide triphosphate to the primer in sequential cycles, thereby to obtain a temporal pattern of nucleotide incorporation.

23. The method according to claim 22, wherein the primer is a specific primer.

24. The method according to claim 22, wherein the primer is a random primer.

25. The method according to claim 22, further comprising the step of comparing the temporal signal pattern of the nucleic acid molecule to the temporal signal pattern of at least one other nucleic acid molecule.

26. The method according to claim 22, wherein the detecting step is performed using a microscope having a total internal reflecting objective.

27. The method according to claim 22, wherein a nucleic acid is located on a substrate at a specific location.

28. The method according to claim 27, wherein a nucleic acid is deposited onto a surface at an identifiable location.

29. The method according to claim 22, wherein the primer has a specific sequence that will capture nucleic acids from a particular region of the genome.

30. The method according to claim 29, wherein the primer comprises a plurality of oligonucleotide sequences.

31. The method according to claim 25, wherein the comparing step comprises determining whether the nucleic acids are derived from a single organism.

32. The method according to claim 31, wherein the organism is selected from the group consisting of a human, an animal, a plant, and a microorganism.

33. The method according to claim 25, wherein the comparing step comprises determining whether the nucleic acids are derived from a single tissue type.

34. The method according to claim 33, wherein the tissue type is selected from the group consisting of a normal organ tissue, a normal body fluid, a diseased organ tissue, and a diseased body fluid.

35. The method according to claim 22, wherein the method is used to diagnose a disease.

36. The method according to claim 22, wherein at least one of the nucleotide triphosphates is a dideoxy nucleotide triphosphate.

37. The method according to claim 22, wherein the label is detected in real time until a signal pattern representative of 2, 3, 4, 5, 6, 7, 8, 9, 10, 11, 12, 13, 14, 15, or 16 nucleotides is determined.
